(19) United States
(12) Patent Application Publication
(10) Pub. No.: US 2025/0387507 A1
(43) Pub. Date: Dec. 25, 2025

(54) HERBAL POLYSACCHARIDE-EXTRACELLULAR VESICLE COMPLEX FOR MAINTAINING RETINAL THICKNESS AND REPAIRING OPTIC NERVE FUNCTION, AND ITS PREPARATION METHOD AND APPLICATION

(71) Applicant: BIOZYME BIOTECHNOLOGY CORP., Banqiao City (TW)

(72) Inventors: CHENG HUANG, Banqiao City (TW); YI-CHUNG LAI, Banqiao City (TW); PO-JU LAI, Banqiao City (TW); PING-CHENG CHAN, Banqiao City (TW)

(21) Appl. No.: 18/896,447

(22) Filed: Sep. 25, 2024

(30) Foreign Application Priority Data
Jun. 20, 2024 (TW) 113122951

(51) Int. Cl. A61K 47/69; A61K 35/747; A61K 36/064; A61K 36/185; A61K 36/815; A61K 36/8984; A61P 25/28; A61P 27/02

(52) U.S. Cl. CPC A61K 47/6901; A61K 35/747; A61K 36/064; A61K 36/185; A61K 36/815; A61K 36/8984; A61P 25/28; A61P 27/02

(57) ABSTRACT

A herbal polysaccharide extracellular vesicle complex produced through fermentation, along with its preparation method and application for promoting neural repair and protecting the optic nerve. The herbal polysaccharide extracellular vesicle complex described herein can maintain retinal thickness, thereby preventing the progression of retinal damage caused by diabetes. This complex offers both optic nerve protection and enhanced neural repair.

### CROSS-REFERENCE TO RELATED APPLICATIONS

This application claims priority in Taiwan Patent Application No. 113122951, filed on June. 20, 2024, which is incorporated by reference in its entirety herein.

### FIELD OF THE INVENTION

This disclosure relates to a biological fermentation extracellular vesicle (EV) technology. Through microbial fermentation techniques, polysaccharide-rich herbal plants such as Dendrobium, Pouteria caimito, and goji berries (Lycium chinense) are used to generate a herbal polysaccharide extracellular vesicle (EV) complex from the fermentation broth. This herbal polysaccharide extracellular vesicle (EV) complex can maintain retinal thickness, thereby preventing the deterioration of retinal damage caused by diabetes. It has neuroprotective functions and can promote nerve repair.

### BACKGROUND OF THE INVENTION

Extracellular vesicle (EV) has been confirmed to serve as carriers with low immunogenicity and high bioavailability, making them easily absorbed and utilized by the human body. They can transport drug components and, due to their small size, have high bioavailability. EVs are also found in microorganisms and plants. EVs are small vesicles with a bilayer lipid membrane structure secreted by living cells into the surrounding environment. They are mainly categorized into exosomes, microvesicles (also known as microparticles), apoptotic bodies, and oncosomes. Additionally, they can be further classified into ectosomes, synaptic vesicles, and matrix vesicles. EVs can be produced by complex eukaryotes, including Gram-negative bacteria, Gram-positive bacteria, mycobacteria, and fungi. Virtually all cells are capable of releasing extracellular vesicle.

Currently, vision health supplements primarily focus on phytochemicals for vision health, such as lutein and zeaxanthin, which function mainly as antioxidants to help photoreceptor cells resist light damage and prevent cell damage caused by excessive oxidation. Vision health can be divided into two aspects: 1) Supplementation of phytochemicals for vision health and 2) optic nerve protection and repair. Optic nerve repair materials can be categorized based on their function into nerve protective materials and nerve repair materials. Optic nerve protective materials are mostly polysaccharides and can be used as food supplements. Nerve repair materials primarily consist of pharmaceutical agents, such as stem cell components or growth-promoting factors like fibroblast growth factors. However, these are currently only used for medical purposes.

Optic nerve health requires both protection and repair. Currently, consumers can only choose food products that protect the optic nerve, while optic nerve repair is primarily achieved through medical means. The vision health market is substantial, with over 90% focused on visual pigment products and the remaining 10% on optic nerve protection. Developing functional ingredients that provide both optic nerve protection and repair would allow for more comprehensive vision health maintenance.

### SUMMARY OF THE INVENTION

In view of the above reasons, present invention utilizes microbial fermentation technology to produce extracellular vesicle (EV). These EVs exhibit high bioavailability and strong tolerance, and can also cross vascular barriers, thereby enhancing the absorption of active ingredients.

The herbal polysaccharide extracellular vesicle complex of present invention simultaneously contains polysaccharides and β-nicotinamide mononucleotide (β-NMN), providing both neuroprotective and neuroregenerative functions for the optic nerve.

The present invention produces extracellular vesicle through a single process. In this process, polysaccharide-rich herbs are fermented, and extracellular vesicle is simultaneously generated. These vesicles inherently contain the necessary active ingredients without the need for additional processing or the incorporation of other complex materials. Polysaccharides and β-NMN are both extracted and generated during the fermentation process.

The present invention develops an ingredient with both neuroprotective and neurorestorative functions in a food-based format. Using extracellular vesicle as carriers, this invention not only protects the optic nerve but also promotes its repair.

The present invention provides a method for preparing an herbal polysaccharide exosome complex, comprising:
(a) An herbal pretreatment step: multiple polysaccharide-rich herbal fruits are separately placed in individual fermentation tanks and ground;
(b) A yeast fermentation step: 0.2˜2% (w/w) of brewing yeast (Saccharomyces cerevisiae) at a concentration of 1*10⁶ CFU/mL is inoculated into each individual fermentation tank, controlling the fermentation environment at 20 Brix sugar content, and a temperature of 25˜28° C., with the addition of 0.01% nicotinamide mononucleotide (NMN) precursor;

(c) A Lactic acid bacteria fermentation step: the contents from the three separate fermentation tanks after yeast fermentation step are mixed in a 1:1:1 ratio and inoculated with 0.22% (w/w) sprouted Lactobacillus plantarum at a concentration of 1*10⁶ CFU/mL, fermenting at 20 Brix sugar content and a temperature of 2528° C. Upon completion of fermentation, the herbal polysaccharide exosome complex can be obtained.

In some embodiments, the polysaccharide-rich herbal fruits comprise Dendrobium, Pouteria caimito, and goji berries (Lycium chinense).

In some embodiments, these polysaccharide-rich herbal fruits are placed in individual fermentation tanks in weight ratios of water to Dendrobium, water to Pouteria caimito, and water to goji berries (Lycium chinense) at 1:1, 10:1, and 10:1 respectively.

Further, the present invention also provides an herbal polysaccharide extracellular vesicle complex, which includes an extracellular vesicle (EV). This extracellular vesicle further comprises a fermented broth of polysaccharide-rich herbal fruits, a polysaccharide, or a β-nicotinamide mononucleotide (β-NMN).

Furthermore, the present invention also provides a method for treating or alleviating nerve damage, comprising administering to the subject an herbal polysaccharide-extracellular vesicle complex.

In some embodiments, the effect of treating or alleviating nerve damage includes promoting the repair of the optic nerve.

Further, the present invention also provides a method for protecting the optic nerve, comprising administering to the subject an herbal polysaccharide extracellular vesicle complex.

In some embodiments, the effect of protecting the optic nerve includes maintaining retinal thickness or preserving the function of ganglion cells.

It is to be understood that both the foregoing general description and the following detailed description are exemplary and explanatory only and are not restrictive of the invention.

### BRIEF DESCRIPTION OF THE DRAWINGS

FIG. 1 shows that the expression of Claudin-5 protein in ganglion cells across different groups in this invention. Values are presented as means±standard deviation. a, P<0.05 indicates a significant difference compared to diabetic retinopathy mice (DR); b, P<0.05 indicates a significant difference compared to the group containing only polysaccharide extracts; c, P<0.05 indicates a significant difference compared to the group containing only β-NMN.

FIG. 2 shows that the expression of Occludin protein in ganglion cells across different groups in this invention is shown, with values presented as means±standard deviation. a, P<0.05 indicates a significant difference compared to diabetic retinopathy mice (DR); b, P<0.05 indicates a significant difference compared to the group containing only polysaccharide extracts; c, P<0.05 indicates a significant difference compared to the group containing only β-NMN.

FIG. 3 shows that the expression of ZO-1 protein in ganglion cells across different groups in this invention is shown, with values presented as means±standard deviation. a, P<0.05 indicates a significant difference compared to diabetic retinopathy mice (DR); b, P<0.05 indicates a significant difference compared to the group containing only polysaccharide extracts; c, P<0.05 indicates a significant difference compared to the group containing only β-NMN.

FIG. 4 shows that the expression of SREBO2 protein in neuronal cells was presented as mean±standard deviation for each group in this invention. a, P<0.05 indicates significant difference compared to diabetic retinopathy (DR) mice; b, P<0.05 indicates significant difference compared to polysaccharide extract alone; c, P<0.05 indicates significant difference compared to β-NMN alone.

FIG. 5 shows that the expression of HMGCR protein in neuronal cells was represented as mean±standard deviation for each group in this invention. a, P<0.05 indicates significant difference compared to diabetic retinopathy (DR) mice; b, P<0.05 indicates significant difference compared to polysaccharide extract alone; c, P<0.05 indicates significant difference compared to β-NMN alone.

FIG. 6 shows that the expression of SIRT1 protein in neuronal cells in each group of present invention.

FIG. 7 shows that the histological staining results of eye tissue slices from different experimental groups in present invention.

FIG. 8 shows that the effect of herbal polysaccharide exosome complex (EVs-NMN) from different experimental groups on the enzyme concentration of superoxide dismutase (SOD) in serum in present invention.

FIG. 9 shows that the effect of herbal polysaccharide exosome complex (EVs-NMN) from different experimental groups on the concentration of glutathione (GSH) in serum in this invention.

FIG. 10 shows that the mechanism of action and effect of EVs-NMN in the present invention.

### DETAILED DESCRIPTION OF THE INVENTION

### Definition

“Lycium chinense” in the present invention refers to goji berries.

Additional specific embodiments of the present invention include, but are not limited to the following:

### EXAMPLE 1

### Preparation of the Herbal Polysaccharide Exosome Complex

(a) An herbal pretreatment step: Pouteria caimito, Dendrobium stems, and goji berries are separately prepared in individual fermentation tanks at water to fruit weight ratios of 1:1, 10:1, and 10:1, respectively. Subsequently, each set of herbal fruits from the individual fermentation tanks is ground using grinding equipment;
(b) A yeast fermentation step: In three separate fermentation tanks, 0.5% (w/w) of brewing yeast strain Saccharomyces cerevisiae, at a concentration of 1*10⁶ CFU/mL, is inoculated. Subsequently, the fermentation sugar content is adjusted to 20 Brix, with fermentation temperature controlled at 25˜28° C. Finally, 0.01% nicotinamide is added as a precursor to NMN. After 14 days of fermentation, Saccharomyces cerevisiae can produce 0.5˜3% β-NMN;
(c) A Lactic acid bacteria fermentation step: Subsequently, the contents of the three fermentation tanks, after the yeast fermentation step, are mixed in a 1:1:1 ratio. Then, 0.5% (w/w) of the fermentation strain Lactobacillus plantarum, at a concentration of 1*10⁶ CFU/mL, is inoculated. The fermentation sugar content is adjusted to 20 Brix, and the fermentation temperature is controlled at 25˜28° C. After 30 days of fermentation, Lactobacillus plantarum undergoes cell division during the fermentation process, forming a large number of exosomes.

### EXAMPLE 2

### Establishment of the Animal Model and Group Descriptions in This Invention

After establishing the diabetic retinopathy (DR) animal model, the diabetic mice were divided into five male groups, including:
Control group (WT group): non-diabetic;
Diabetic retinopathy control group (DR group): diabetic retinopathy;
Low dose group (LD group): induced diabetes and administered a low dose (0.195 mg per gram of mouse body weight) of the fermentation complex or polysaccharide extract or β-NMN;
Recommended dose group (MD group): induced diabetes and administered a recommended dose (0.39 mg per gram of mouse body weight) of the fermentation complex or polysaccharide extract or β-NMN;
High dose group (HD group): induced diabetes and administered a high dose (1.17 mg per gram of mouse body weight) of the fermentation complex or polysaccharide extract or β-NMN.

Each group and each test sample included 12 mice. For the groups administered the fermentation complex, the fermentation complex, polysaccharide extract, or β-NMN were continuously administered via gavage for 28 days.

EXAMPLE 3

### Expression of Neurotrophic Proteins in Retinal Ganglion Cells

In this experiment, the animal model established in Example 2 was used. Retinal cell sampling was subsequently performed on the mice to analyze the expression of SIRT1, SREBP2, and HMGCR proteins.

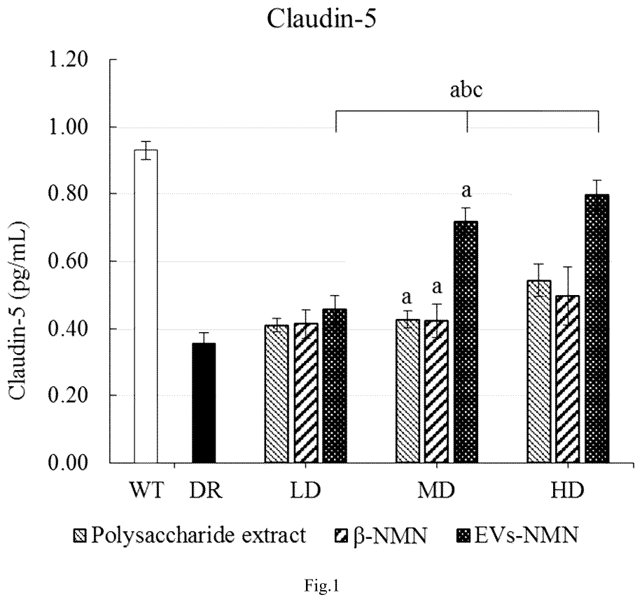

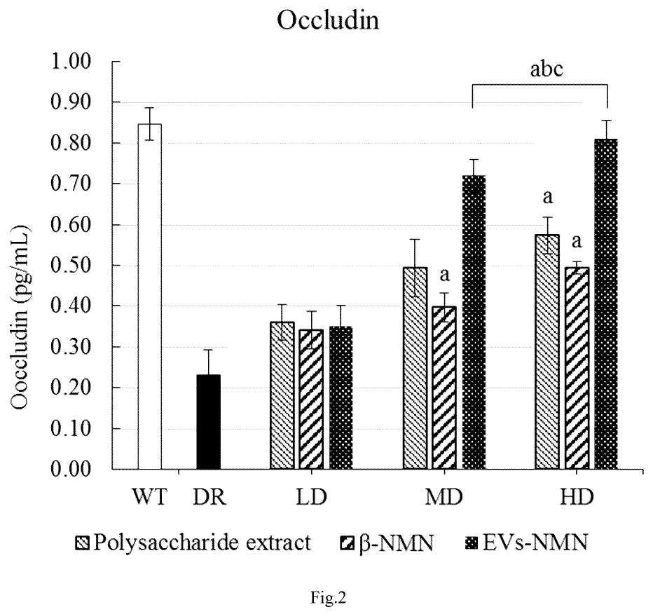

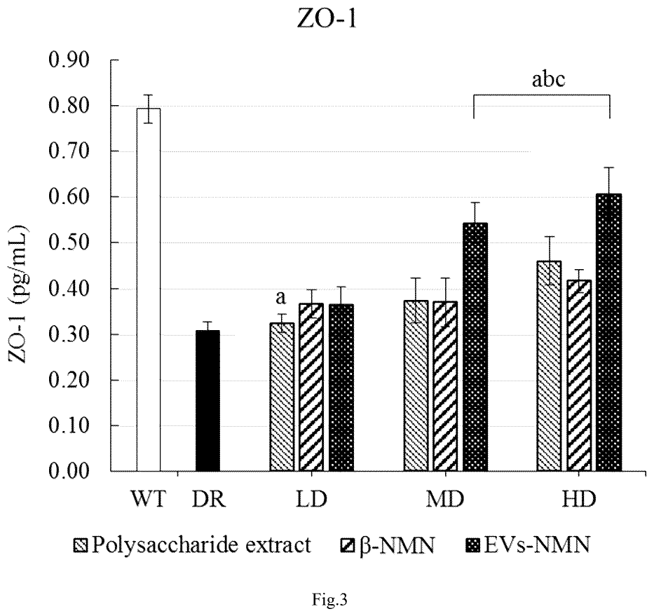

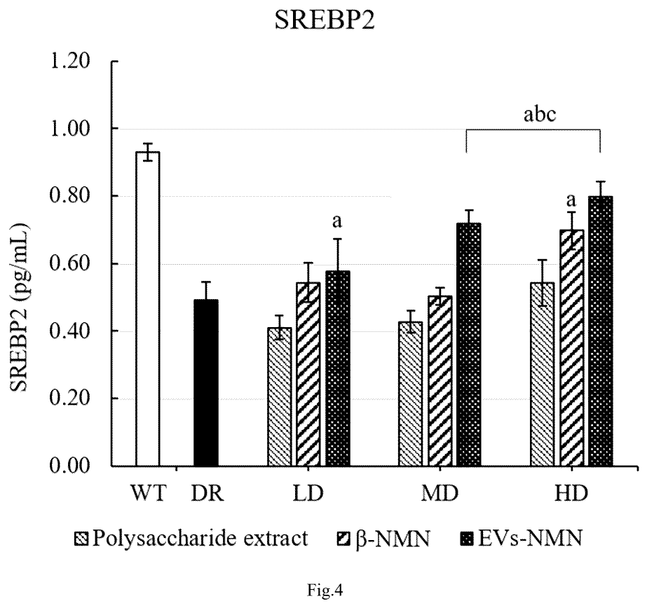

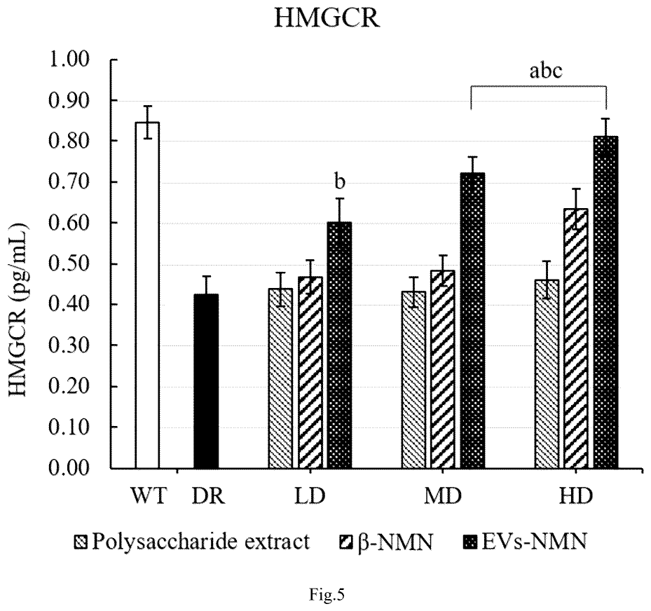

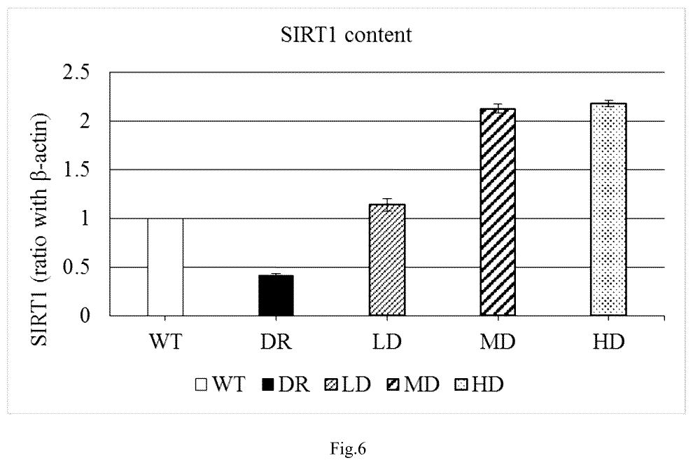

The results are shown in FIGS. 1 to 6. The DR group exhibited oxidative stress damage to the retina. This oxidative damage led to an increase in the inflammatory factor TNF-α, which promoted leukocyte adhesion to the vascular walls, subsequently obstructing the blood-retinal barrier (BRB). When nutrients and blood cannot pass through to the retina, retinal cell damage occurs.

The repair of the blood-retinal barrier (BRB) relies on the formation of tight junction proteins, with the increase of Claudin-5, Occludin, and ZO-1 proteins promoting BRB repair, thereby maintaining the normal morphology of retinal ganglion cells. Experimental results show that the herbal polysaccharide exosome complex (EVs-NMN) in the recommended dose group and the high dose group significantly increases protein expression. Compared to the use of polysaccharide extract or β-NMN alone, there are also significant differences, indicating that EVs-NMN has a role in promoting retinal ganglion cell repair.

Additionally, based on the retinal ganglion cell repair pathway, studies have indicated that increased expression of the SIRT1 protein can promote an increase in cholesterol concentration in ganglion cells, which aids in DNA repair and enhances the expression of SREBP2 and HMGCR. This pathway can increase the number of retinal ganglion cells (RGCs). The analysis results show that the use of EVs-NMN promotes a significant increase in SIRT1 protein expression in retinal cells, along with elevated levels of SREBP2 and HMGCR expression. Therefore, it can help increase the number of RGCs, achieving the effect of repairing visual nerve cells.

### EXAMPLE 4

### Histological Staining Experiment of Eye Tissue

This experiment utilized the animal model established in Example 2. After 28 days of feeding, mice were sacrificed, their eyes were sampled, and eye tissue sections were stained.

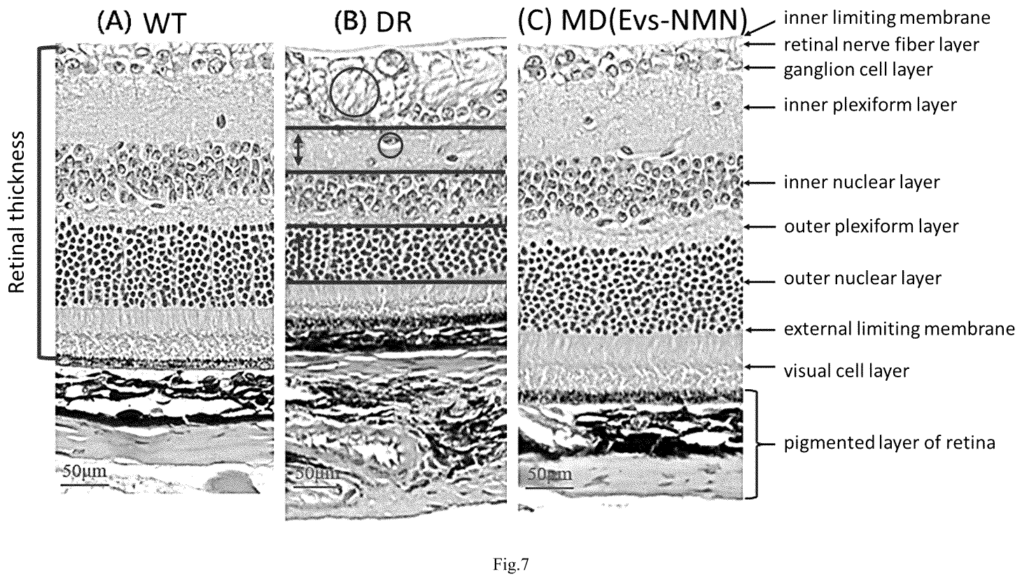

As shown in FIG. 7, Part B represents mice from the control group, exhibiting significant vacuolization and neovascularization in the ganglion cell layer. The inner plexiform layer also shows vacuolization and thinning, while the inner nuclear layer appears disorganized with noticeable thinning. The outer nuclear layer also exhibits thinning. These findings indicate that prolonged hyperglycemia can lead to retinal thinning, damage to retinal ganglion cells, and an increased risk of blindness. Part C represents mice from the recommended dose group. Results show that after administration of EVs-NMN at the recommended dose, there is significant improvement in ganglion cell vacuolization, with a gradual improvement in ganglion cell arrangement. The inner plexiform layer, inner nuclear layer, and outer nuclear layer show trends towards thickening, with a more organized structure. This demonstrates that EVs-NMN can improve retinal thinning, protect retinal layer thickness, facilitate recovery of damaged ganglion cells, and prevent retinal vascular proliferation.

### EXAMPLE 5

### Analysis of Optic Nerve/Retina Antioxidant Protective Function

This experiment utilized the animal model established in Example 2, blood samples were collected from mice in the control group, reference group, low-dose group, standard-dose group, and high-dose group to analyze the enzyme concentrations of superoxide dismutase (SOD) and glutathione (GSH) in serum.

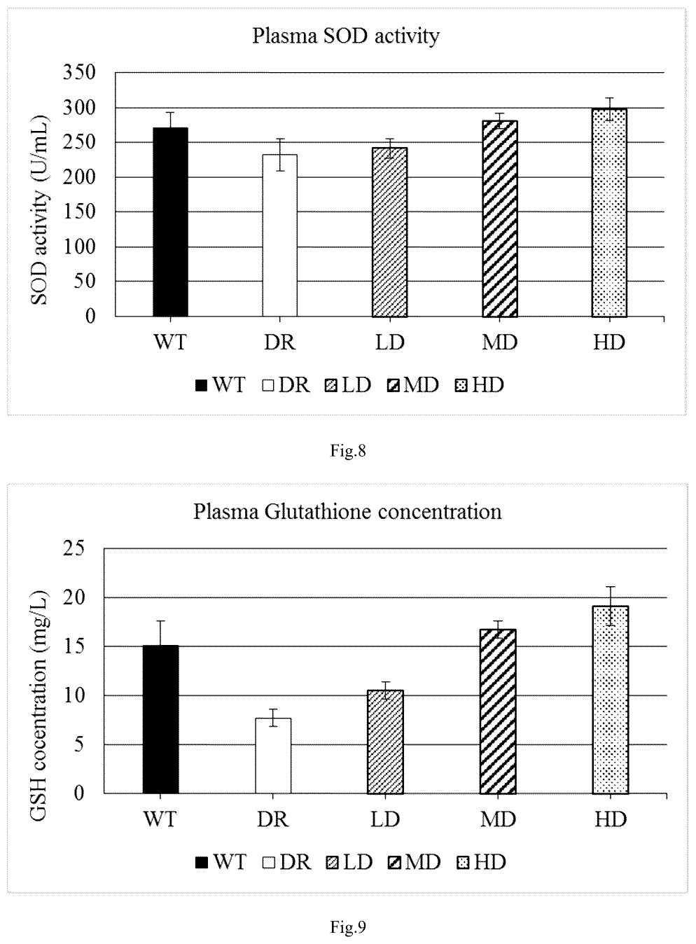

As shown in FIGS. 8 to 9, research indicates that retinal diseases caused by light damage or high glucose levels lead to a decrease in intracellular concentrations of SOD or antioxidant substances, thereby damaging retinal cells and causing thinning of the retina, impairing vision. In diabetic retinopathy mice treated with EVs-NMN according to the present invention, the concentrations of SOD and GSH in their blood significantly increased. These results suggest that EVs-NMN enhances retinal antioxidant capacity, thereby protecting retinal and neuronal functions.

### EXAMPLE 6

### The Main Functional Mechanism of This Invention's Herbal Polysaccharide Extracellular Vesicle Complex

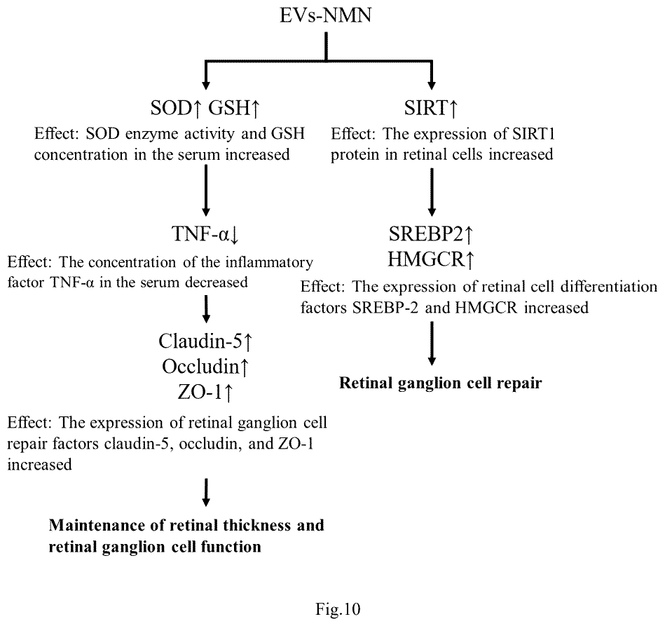

The mechanism of action of EVs-NMN in the present invention is shown in FIG. 10.
1. EVs-NMN enhances the concentrations of SOD and GSH, thereby mitigating the downregulation of TNF-α expression. This leads to increased expression of retinal ganglion cell repair factors, such as Claudin-5, Occludin, and ZO-1, which ensures the maintenance of retinal thickness and preservation of retinal ganglion cell function.
2. EVs-NMN upregulates the expression of SIRT proteins, which in turn increases the levels of retinal cell differentiation factors SREBP2 and HMGCR, thereby facilitating retinal ganglion cell repair.

All examples provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority or inferiority of the invention. Although one or more embodiments of the present invention have been described in detail, it should be understood that the various changes, substitutions, and alterations could be made hereto without departing from the spirit and scope of the invention.

It is intended that the specification and examples be considered as examples only, with a true scope and spirit of the invention being indicated by the following claims.

1. A method for preventing aan herbal polysaccharide exosome complex, comprising:
(A) a herbal pretreatment step: multiple polysaccharide-rich herbal fruits are separately placed in individual fermentation tanks and ground;
(B) a yeast fermentation step: 0.2˜2% (w/w) of brewing yeast (Saccharomyces cerevisiae) at a concentration of 1*10⁶ CFU/mL is inoculated into each individual fermentation tank, controlling the fermentation environment at 20 Brix sugar content, and a temperature of 25˜28° C., with the addition of 0.01% nicotinamide mononucleotide (NMN) precursor; and
(C) a Lactic acid bacteria fermentation step: the contents from the three separate fermentation tanks after yeast fermentation are mixed in a 1:1:1 ratio and inoculated with 0.22% (w/w) sprouted Lactobacillus plantarum at a concentration of 1*10⁶ CFU/mL, fermenting at 20 Brix sugar content and a temperature of 25˜28° C., upon completion of fermentation, the herbal polysaccharide exosome complex can be obtained.

2. The method of claim 1, wherein the polysaccharide-rich herbal fruits comprise Dendrobium, Pouteria caimito, and goji berries (Lycium chinense).

3. The method of claim 1, wherein the polysaccharide-rich herbal fruits are placed in individual fermentation tanks in weight ratios of water to Dendrobium, water to Pouteria caimito, and water to goji berries (Lycium chinense) at 1:1, 10:1, and 10:1 respectively.

4. A herbal polysaccharide extracellular vesicle complex, comprising an extracellular vesicle (EV), wherein the extracellular vesicle (EV) further comprises a fermented broth of polysaccharide-rich herbal fruits, a polysaccharide, or a β-nicotinamide mononucleotide (β-NMN).

5. The herbal polysaccharide extracellular vesicle complex of claim 4, wherein the polysaccharide-rich herbal fruits comprise Dendrobium, Pouteria caimito, and goji berries (Lycium chinense).

6. A method for treating or alleviating nerve damage, comprising administering to the subject the herbal polysaccharide extracellular vesicle complex of claim 4.

7. The method of claim 6, wherein the effect of treating or alleviating nerve damage includes promoting the repair of the optic nerve.

8. A method for protecting the optic nerve, comprising administering to the subject the herbal polysaccharide extracellular vesicle complex of claim 4.

9. The method of claim 8, wherein the effect of protecting the optic nerve includes maintaining retinal thickness or preserving the function of ganglion cells.
